(19) United States
(12) Patent Application Publication
(10) Pub. No.: US 2013/0188819 A1
(43) Pub. Date: Jul. 25, 2013

(54) EAR TIP AND EAR PHONE HAVING THE SAME

(71) Applicant: Yoon Young-Mun, Seongnam-si (KR)

(72) Inventor: Yoon Young-Mun, Seongnam-si (KR)

(73) Assignee: ALPHA PRECISION CO., LTD., Yongin-si (KR)

(21) Appl. No.: 13/405,884

(22) Filed: Feb. 27, 2012

(30) Foreign Application Priority Data
Jan. 20, 2012 (KR) 10-2012-0006481

(51) Int. Cl. H04R 1/10; A61F 11/08

(52) U.S. Cl. 381/380; 181/129

(57) ABSTRACT

In an ear tip and an ear phone including the same, the ear tip includes a cylindrical hollow shaft having a sound conduit through which audio signals are transferred into a user's ear, an external sheet extending from an end portion of the hollow shaft and enclosing the hollow shaft such that a gap space is provided between the hollow shaft and the external sheet, and a reinforce member protruded from the external sheet toward the hollow shaft in the gap space and reinforcing a strength of the external sheet to thereby resist against an axial external force applied along the hollow shaft. Accordingly, the external sheet is prevented from being turned inside out by the axial external force.

### CROSS-REFERENCE TO RELATED APPLICATIONS

This application claims the benefit of Korean Patent Application No. 10-2012-0006481 filed on Jan. 20, 2012 in the Korean Intellectual Property Office, the disclosure of which is incorporated herein in its entirety by reference.

### BACKGROUND

1. Field

Example embodiments relate to an ear tip and an ear phone having the same, and more particularly, to an ear tip for an in-ear electro-acoustic transducer (in-ear headphone or ear phone) and an ear phone having the same.

2. Description of the Related Art

Various headphones have been used for listening audio signals in a single mode or for listening high quality of the audio signals. Particularly, as mobile devices such as a personal media player (PMP), a smart phone and a tablet PC have been widely used in a recent time, there have been plenty of chances and needs for individually listening the audio signals such as many pieces of music and lecture files without any external noises and disturbances from surroundings. For those reasons, high sealed and fidelity headphones are now in great demand.

In general, the headphones may be classified into an around-ear headphone covering a whole ear of the users, an on-ear headphone arranged on the user's ear and an in-ear headphone inserted into an external ear of the users. The above wide spreads of the mobile devices rapidly increase the demand for the in-ear headphone, which is usually called as an ear phone, due to the excellent portability.

Conventional ear phones includes a body for converting electrical signals to sounds or the audio signals and an ear tip detachably coupled to the body and making contact with an ear skin of the users. The body usually comprises hard materials such as hard polymer and metal and includes some grooves and stepped portions at an end portion for reinforcing the coupling between the body and the ear tip.

The ear tip usually includes a sound conduit coupled to the groove and the stepped portion of the body and transferring the sound into the user's ear therethrough and a external sheet extending from an end portion of the sound conduit and surrounding the sound conduit in such a configuration that an ear hole or an auditory canal of the user's ear makes close contact with the external sheet. Thus, an internal ear and an external ear are separated from each other in the user's ear and the environmental noises are usually prevented from being transferred into the internal ear from surroundings. Since the external sheet makes direct contact with the user's skin in the ear, the external sheet usually plays a key role for comfortable and natural usage and high degree of noise-proofing of the ear phone.

Conventionally, the external sheet of the ear phone usually comprises flexible materials such as silicon or rubber based materials and thus is likely to be deformed in accordance with the shape of the auditory canal of the user's ear. Particularly, the external sheet needs to make close contact with the skin of the ear hole so as to prevent the noise transfer and the separation from the ear due to the slip on the skin.

When the users may apply external forces to the ear phone so as to separate the ear phone out of the ear, the external sheet of the ear phone tends to be drawn toward the internal ear due to the friction force between the external sheet and the skin of the ear hole. Since the external sheet of the conventional ear phone does not have sufficient strength and roughness enough for resisting the frictional force due to a very small thickness thereof and the sound conduit of the ear tip is strongly coupled to the body of the body of the ear phone by the stepped portion, the external sheet of the ear tip is to frequently be turned inside out when separating the ear phone from the user's ear. The external sheet is much more frequently tuned inside out as the skin and the external sheet is more closely contact with each other.

In addition, the external sheet of the ear tip usually tends to be turned inside out by a slight external force even though the ear phone to which the ear tip is attached is under a non-working state out of the user's ear, and thus the external sheet frequently needs to be returned into the original shape before the ear phone is inserted into the user's ear. As a result, the external sheet is likely to be broken by the repetition of the turning inside out and the return to the original shape.

Accordingly, there is still a need for an improved ear tip for the ear phone having a sufficient strength for resisting the turning inside out of the cover sheet.

### SUMMARY

Example embodiments of the present inventive concept provide an ear tip having a reinforce member for reinforcing the strength and roughness of the cover sheet.

Example embodiments of the present inventive concept also provide an ear phone including the above ear tip.

According to some example embodiments, there is provided an ear tip including a cylindrical hollow shaft having a sound conduit through which audio signals may be transferred into a user's ear, an external sheet extending from an end portion of the hollow shaft and enclosing the hollow shaft such that a gap space may be provided between the hollow shaft and the external sheet, and a reinforce member protruded from the external sheet toward the hollow shaft in the gap space and reinforcing a strength of the external sheet to thereby resist against an axial external force applied along the hollow shaft.

In an example embodiment, the hollow shaft, the external sheet and the reinforce member may include same materials and the hollow shaft, the external sheet and the reinforce member may comprise silicon having a hardness of 20° to 45°.

In an example embodiment, the reinforce member may include one of the curved plate, a flat plate a cone and a waved plate that may be protruded from an inner surface of the external sheet.

In an example embodiment, the hollow shaft may comprise a material different from the external sheet and the hollow shaft and the external sheet are individually connected to each other. The hollow shaft may comprise silicon having hardness of 50° to 60° and the external sheet may comprise silicon having hardness of 10° to 15°. The reinforce member may comprise silicon having hardness of 20° to 45°.

In an example embodiment, the ear tip may further comprise a noise absorber arranged around the end portion of the hollow shaft in the gap space. The noise absorber may absorb environmental noises and preventing noise transfer into the user's ear. The noise absorber may include an elastic gel state material having hardness of 5° to 20°.

According to some example embodiments, there is provided an ear phone including the above ear tip. The ear phone may include a housing including an audio signal generator, a cover detachably coupled to the housing such that the housing may be covered with the cover and an inner space of the housing may be closed from surroundings and an ear tip detachably attached to the cover and guiding the audio signals into the user's ear. The cover may include a sound guide through which the audio signal may be discharged out of the closed inner space. The ear tip may include a cylindrical hollow shaft having a sound conduit through which the audio signals may be transferred into a user's ear, an external sheet extending from an end portion of the hollow shaft and enclosing the hollow shaft such that a gap space may be provided between the hollow shaft and the external sheet, and a reinforce member protruded from the external sheet toward the hollow shaft in the gap space and reinforcing a strength of the external sheet to thereby resist against an axial external force applied along the hollow shaft.

In an example embodiment, the sound guide may be shaped into a tube having at least a recess and at least a protrusion at an end portion thereof and the hollow shaft of the ear tip may include a stepped unit having a receiving space in which the protrusion may be received in such a configuration that the protrusion and the stepped unit may make surface contact with each other in an axial direction of the hollow shaft.

According to example embodiments of the present inventive concept, the reinforce member may be provided in the gap space S between the hollow shaft including the sound conduit and the external sheet enclosing the hollow shaft, and thus the strength of the external sheet may be reinforced by the reinforce member along the axial direction of the hollow shaft. Accordingly, the external sheet may be prevented from being turned inside out by the axial external direction, to thereby prevent the breakage of the ear tip and increase the durability of the ear tip. In addition, the noise absorber may be further provided around the bottom of the hollow shaft in the gap space, to thereby efficiently prevent the noise transfer into the ear and increase sound quality. Particularly, when the hollow shaft of the ear tip may comprise a relatively hard material and the external sheet of the ear tip may comprise a relatively soft material, both of the contact stability and the sound quality of the ear phone may be improved due to the material properties of the ear tip.

### BRIEF DESCRIPTION OF THE DRAWINGS

Example embodiments will be more clearly understood from the following detailed description taken in conjunction with the accompanying drawings.

FIG. 1A is a perspective view illustrating an ear tip in accordance with an example embodiment of the present inventive concept;

FIG. 1B is a cross-sectional view of the ear tip shown in FIG. 1A;

FIG. 2A is a perspective view illustrating a first modification of the reinforce member shown in FIG. 1A;

FIG. 2B is a cross-sectional view of the first modified reinforce member shown in FIG. 2A;

FIG. 3A is a perspective view illustrating a second modification of the reinforce member shown in FIG. 1A;

FIG. 3B is a cross-sectional view of the second modified reinforce member shown in FIG. 3A;

FIG. 4A is a perspective view illustrating a third modification of the reinforce member shown in FIG. 1A;

FIG. 4B is a cross-sectional view of the third modified reinforce member shown in FIG. 4A;

FIG. 5 is a perspective view illustrating an ear tip in accordance with another example embodiment of the present inventive concept;

FIG. 6 is a perspective view illustrating an ear tip in accordance with still another example embodiment of the present inventive concept; and

FIG. 7 is an explosive perspective view illustrating an ear phone including an ear tip in accordance with an example embodiment of the present inventive concept.

### DETAILED DESCRIPTION OF THE EMBODIMENTS

Various example embodiments will be described more fully hereinafter with reference to the accompanying drawings, in which some example embodiments are shown. The present invention may, however, be embodied in many different forms and should not be construed as limited to the example embodiments set forth herein. Rather, these example embodiments are provided so that this disclosure will be thorough and complete, and will fully convey the scope of the present invention to those skilled in the art. In the drawings, the sizes and relative sizes of layers and regions may be exaggerated for clarity.

It will be understood that when an element or layer is referred to as being “on,” “connected to” or “coupled to” another element or layer, it can be directly on, connected or coupled to the other element or layer or intervening elements or layers may be present. In contrast, when an element is referred to as being “directly on,” “directly connected to” or “directly coupled to” another element or layer, there are no intervening elements or layers present. Like numerals refer to like elements throughout. As used herein, the term “and/or” includes any and all combinations of one or more of the associated listed items.

It will be understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular example embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Example embodiments are described herein with reference to cross-sectional illustrations that are schematic illustrations of idealized example embodiments (and intermediate structures). As such, variations from the shapes of the illustrations as a result, for example, of manufacturing techniques and/or tolerances, are to be expected. Thus, example embodiments should not be construed as limited to the particular shapes of regions illustrated herein but are to include deviations in shapes that result, for example, from manufacturing. Thus, the regions illustrated in the figures are schematic in nature and their shapes are not intended to illustrate the actual shape of a region of a device and are not intended to limit the scope of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Hereinafter, example embodiments will be explained in detail with reference to the accompanying drawings.

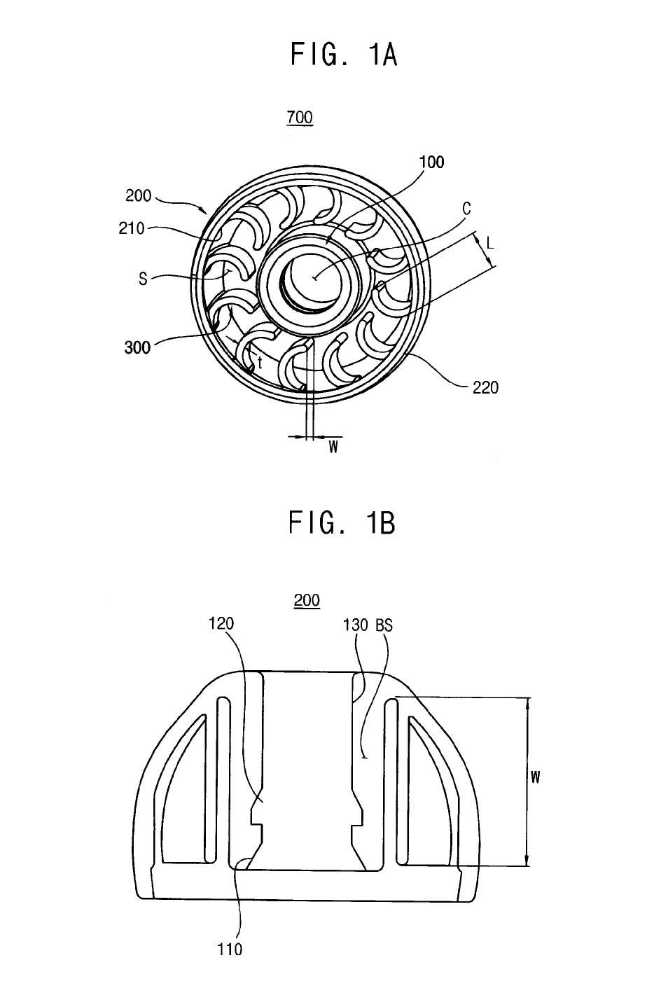

FIG. 1A is a perspective view illustrating an ear tip in accordance with an example embodiment of the present inventive concept and FIG. 1B is a cross-sectional view of the ear tip shown in FIG. 1A.

Referring to FIGS. 1A and 1B, an ear tip 700 in accordance with an example embodiment of the present inventive concept may include a cylindrical hollow shaft 100 having a sound conduit C through which audio signals may be transferred, an external sheet 200 extending from an end portion of the hollow shaft 100 and enclosing the hollow shaft 100 in such a configuration that a gap space S may be provided between the hollow shaft 100 and the external sheet 200 and a reinforce member 300 protruded from the external sheet 200 toward the hollow shaft 100 in the gap space S to thereby reinforce a strength of the external sheet 200 and to sufficiently resist against an external force applied to the external sheet 200 along the hollow shaft 100.

In an example embodiment, the hollow shaft 100 may be shaped into a liner cylinder having a predetermined length and an opening may be provided through the hollow shaft 100 through which the audio signals may be transferred. The opening passing through the hollow shaft 100 may function as the sound conduit C. Thus, a first end portion of the hollow shaft 100 may be coupled to a body of an ear phone (not illustrated) in which the audio signals may be generated from electrical signals, and a second end portion opposite to the first end portion of the hollow shaft 100 may be inserted into a user's ear. Thus, the sound conduit C may be communicated with an inner space of the user's ear.

For example, the hollow shaft 100 may include a guide unit 110 for guiding a connector unit (not illustrated) of the ear phone body to the hollow shaft 100, a stepped unit 120 to which the connector unit of the ear phone body may be coupled to thereby prevent the separation of the hollow shaft 100 and the connector unit and a conduit body 130 for transferring the audio signals into the user's ear.

In the present example embodiment, the guide unit 110, the stepped unit 120 and the conduit body 130 may be sequentially connected with one another and be integrally formed into one body. The guide unit 110 may be shaped into a trapezoidal cylinder in which cross-sectional circular surfaces may have different diameters along the central axis of the hollow shaft 100. Thus, the connector unit of the ear phone body may smoothly slide into the guide unit 110 at the first end portion of the hollow shaft 100 and may be pushed along the guided unit 110 until the connector unit may be coupled to the stepped unit 120. The stepped unit 120 may be shaped into a cylinder of which the diameter of the cross-sectional circular surface may be larger than those of the neighboring the guide unit 110 and the conduit body 130. Therefore, once the connector unit of the ear phone body may be coupled to the stepped portion 120 of the hollow shaft 100, the hollow shaft 100 and the connector unit of the ear phone body may be difficult to be separated from each other. That is, the connector unit of the ear phone body may be separated from the hollow shaft 100 just merely on condition that an external force may be applied to the ear phone body and the ear tip 700 over the frictional force between the stepped unit 120 and the connector unit of the ear phone body. The conduit body 130 may determine an overall shape of the hollow shaft 100 and have a sufficient hardness for reflecting the audio signals even though the audio signals or the sounds may have a relatively high frequency. The conduit body 130 may be connected to the external sheet 200 at the second end portion of the hollow shaft 100.

The external sheet 200 may extend from an end portion of the conduit body 130 toward the guide unit 110 and may be spaced apart from the hollow shaft 100 by a gap distance in such a configuration that the hollow shaft 100 may be enclosed by the external sheet 200. Thus, the gap space S may be provided between the hollow shaft 100 and the external sheet 200. Since the external sheet 200 may be connected with the hollow shaft 100 at the second end portion, the gap space S may be closed from surroundings around the second end portion of the hollow shaft 100 and may be open around the first end portion of the hollow shaft 100. That is, the external sheet 200 may be shaped into a pot in which the hollow shaft 100 may be positioned at a central portion thereof. In addition, the external sheet 200 may make contact with the skin in the user's ear and thus may have a profile corresponding to a normal ear hole.

In the present example embodiment, the external sheet 200 may have the same materials as the hollow shaft 100. For example, the external sheet 200 may comprise a soft silicon gel or a rubber to thereby improve contact stability with respect to the skin and reduce dissimilarity in the ear hole. The external sheet 200 and the hollow shaft 100 may have the same material and may be integrally formed in one body. Particularly, the hollow shaft 100 may have a thickness larger than that of the external sheet 200, thus the sound may be sufficiently well reflected from the sound conduit C and the dissimilarity between the skin and the external sheet 200 may be minimized in the ear hole. In the present example embodiment, the hardness of the external sheet 200 may be in a range of about 20° to about 45° and the hollow shaft 100 may also have the same hardness.

The reinforce member 300 may be protruded from an inner surface 210 of the pot-shaped external sheet 200 toward the hollow shaft 100 in the gap space S. Particularly, the reinforce member 300 may not make contact with the hollow shaft 100 so as not to deteriorate the flexibility of the external sheet 200.

For example, the reinforce member 300 may have a length L measured from the inner surface 210 of the external sheet 200, a width W measured in a direction along the hollow shaft 100 and a thickness t measured in a direction along a circumferential line around the hollow shaft 100. Particularly, the thickness of the reinforce member 300 may be sufficiently small as compared with the length L and the width W, thus the reinforce member 300 may be shaped into a plurality of curved sheet arranged in the circumferential line around the hollow shaft 100. Therefore, the reinforce member 300 may be sufficiently flexible and freely deformed according to the external forces applied thereto in a circumferential direction and in a radial direction of the hollow shaft 100. That is, the reinforce member 300 may not resist against the external force along the circumferential and radial directions of the hollow shaft 100. In contrast, the reinforce member 300 may have a sufficient strength against the external force applied thereto in an axial direction of the hollow shaft 100, and thus the reinforce member 300 may hardly deformed by the axial external force along the hollow shaft 100. That is, the reinforce member 300 may sufficiently reinforce the strength of the external sheet 200 against the external force in the axial direction of the hollow shaft 100.

When the connector unit of the ear phone body may be coupled to or decoupled from the hollow shaft 100 of the ear tip 700 and the ear phone including the ear tip 700 may be inserted into or drawn back out of the ear hole of the user's ear, the external sheet 200 of the war tip 700 may be sufficiently resistive against the axial external force applied by the user substantially along the central axis of the hollow shaft 100, thereby preventing the external sheet 200 from being turned inside out.

In the present example embodiment, the reinforce member 300 may have the same materials as the external sheet 200 and may be integrally formed together with the external sheet 200 in one body. Therefore, the hollow shaft 100, the external sheet 200 and the reinforce member 300 may comprise the same material such as a silicon gel of which the hardness may be in a range of about 20° to about 45°. The hollow shaft 100, the external sheet 200 and the reinforce member 300 may be integrally formed together with one another in a body through a single process.

Further, the number of the reinforce members 300 may be varied in accordance with the axial external force along the hollow shaft 100. The strength of the external sheet 200 against the axial external force may be reinforced in proportion to the number of the reinforce members 300. However, the reinforce members 300 on the inner surface of the external sheet 200 may increase the dissimilarity of the ear phone in the ear hole and deteriorate the flexibility of the external sheet 200. Therefore, the number of the reinforce members 300 may be optimized in view of the strength increase and the flexibility deterioration.

While the present example embodiment discloses that the external sheet 200 may have the same materials as the reinforce member 300, the external sheet 200 may have some materials different from those of the reinforce member 300, as would be known to one of the ordinary skill in the art.

The reinforce member 300 may have various shapes in accordance with the shape of the connector unit of the ear phone body and the some usage conditions. Proper modifications to the reinforce member 300 may decrease the number of the reinforce member 300 without any decrease of the strength of the external sheet 200 according to the magnitude and property of the axial external force along the hollow shaft 100.

In the present example embodiment, the reinforce member 300 may be shaped into a concave plate and may be arranged in a clockwise direction around the hollow shaft 100 in the gap space S. Thus, while the reinforce member 300 may be hardly resistive to the radial external force along the radial direction of the hollow shaft 100, the reinforce member 300 may be sufficiently resistive against the axial external force along the axial direction of the hollow shaft 100. Accordingly, the strength of the external sheet 200 may be reinforced as much as the bulk resistive force in proportional to the volume of the reinforce member 300 determined by the length L, the width W and the width t of the reinforce member 300. The external sheet 200 may be sufficiently resistive against the axial external force and thus may be difficult to be turned inside out when the axial external force may be applied to the ear tip 700.

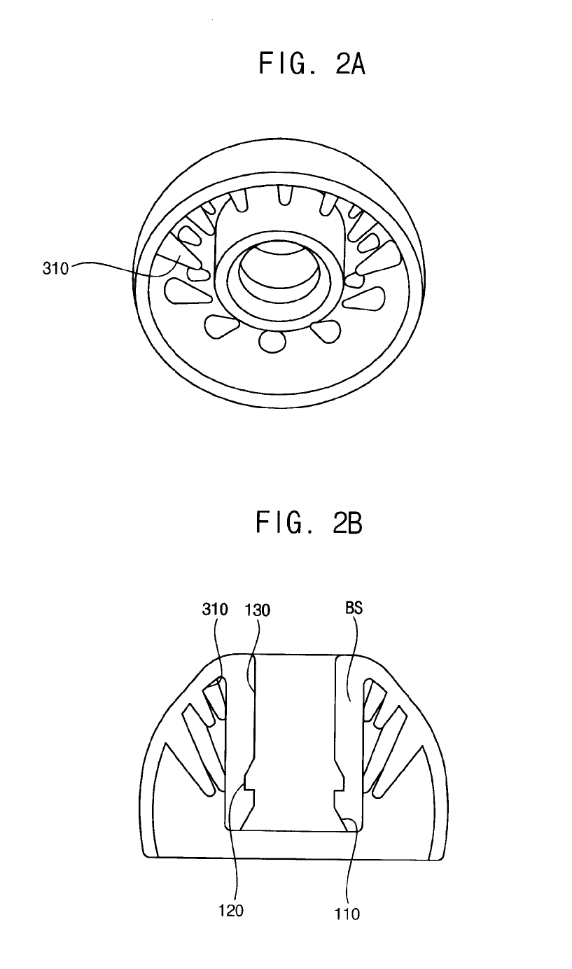

FIG. 2A is a perspective view illustrating a first modification of the reinforce member shown in FIG. 1A and FIG. 2B is a cross-sectional view of the first modified reinforce member shown in FIG. 2A.

Referring to FIGS. 2A and 2B, the first modification 310 of the reinforce member 300 may be shaped into a cone extending from the inner surface 210 of the external sheet 200 to the hollow shaft 100. For example, a plurality of the cone-shaped reinforce members 310 may be uniformly arranged on the inner surface of the external sheet 200 and the strength of the external sheet 200 may be reinforced as much as the bulk resistive force in proportional to the volume of the cone-shaped reinforce member 310.

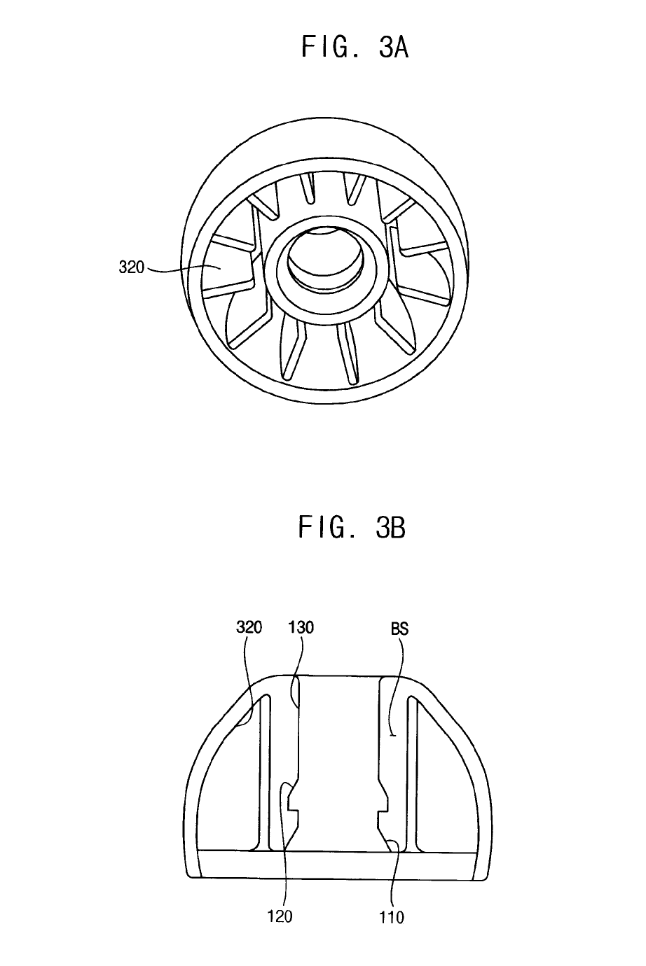

FIG. 3A is a perspective view illustrating a second modification of the reinforce member shown in FIG. 1A and FIG. 3B is a cross-sectional view of the second modified reinforce member shown in FIG. 3A.

Referring to FIGS. 3A and 3B, the second modification 320 of the reinforce member 300 may be shaped into a flat plate extending from the inner surface 210 of the external sheet 200 to the hollow shaft 100. For example, a plurality of the flat reinforce members 320 may be uniformly arranged in the gap space S and thus the gap space S may be uniformly divided into a plurality of space pieces having the same size. Thus, the strength of the external sheet 200 may be reinforced as much as the bulk resistive force in proportional to the volume of the flat reinforce member 320.

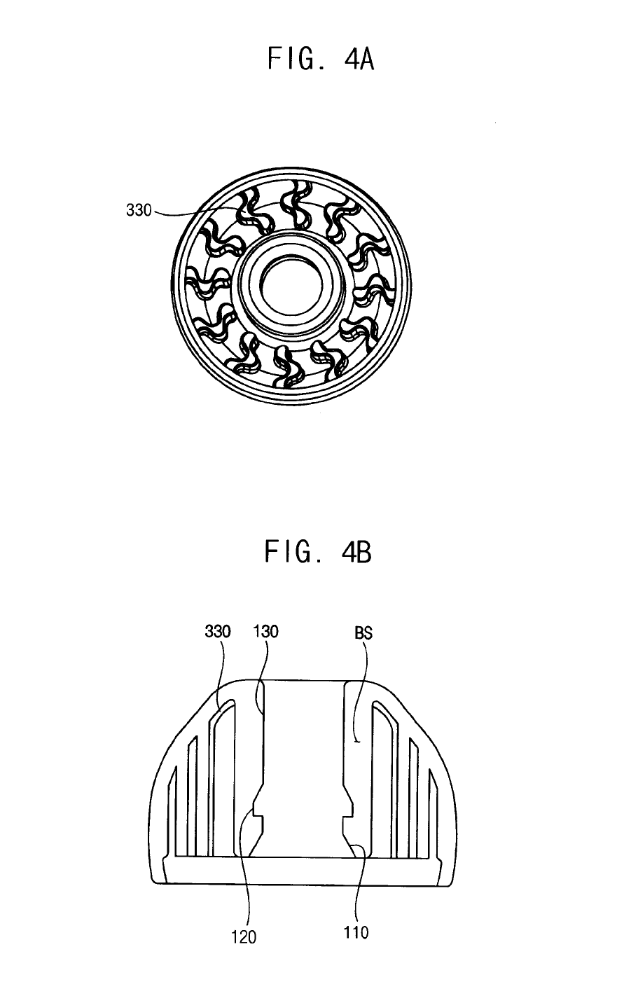

FIG. 4A is a perspective view illustrating a third modification of the reinforce member shown in FIG. 1A and FIG. 4B is a cross-sectional view of the third modified reinforce member shown in FIG. 4A.

Referring to FIGS. 4A and 4B, the third modification 330 of the reinforce member 300 may be shaped into a wave-shaped plate extending from the inner surface 210 of the external sheet 200 to the hollow shaft 100. A plurality of recesses and protrusions may be provided on surfaces of the plate along the length L thereof, and thus the plate extends from the inner surface 210 of the external sheet 200 just like a wave. A plurality of the wave shaped reinforce members 330 may be uniformly arranged in the gap space S and thus the gap space S may be uniformly divided into a plurality of space pieces having the same size. Thus, the strength of the external sheet 200 may be reinforced as much as the bulk resistive force in proportional to the volume of the wave-shaped reinforce member 330.

The modified reinforce members 310 to 330 in FIGS. 2A to 4B may be illustrative of example modifications of the reinforce member 300 and may not be construed as limiting thereof. Thus, any other modifications of the reinforce member 300 may be allowable as long as the modifications of the reinforce member 300 may be sufficiently resistive against the axial external force along the axial direction of the hollow shaft 100 and thus the strength of the external sheet 200 may be sufficiently reinforced due to the modifications of the reinforce member 300.

Particularly, the reinforce member 300 may not make contact with the hollow shaft 100, while extending from the inner surface of the external sheet 200. The reinforce member 300 may be spaced apart from the hollow shaft 100 and thus a buffer space BS may be provided between the reinforce member 300 and the hollow shaft 100. The space pieces of the gap space S may be communicated with one another through the buffer space BS.

When the radial external force may be applied to an outer surface 220 of the external sheet 200, the deformation of the external sheet 200 may be absorbed within the buffer space BS. Thus, the reinforce member 300 may not be resistive against the radial external force applied to the external sheet 200 along the radial direction of the hollow shaft 100. In addition, when the radial external force applied to the external sheet 200 may be over the volume of the buffer space BS, the reinforce member 300 may be arranged into such a configuration that the reinforce member 300 may be sufficiently deformed by the radial external force so as not to deteriorate the flexibility of the ear tip 700 in spite of the reinforce member 300. However, when the axial external force may be applied to the external sheet 200, the deformation of the reinforce member 300 due to the axial external force may be sufficiently minimized and thus may be sufficiently resistive against the axial external force, to thereby reinforce the strength of the external sheet 200.

For example, when the reinforce member 300 may have the length L sufficiently larger than the width W, the deformation of the reinforce member 300 due to the radial external force may be freely allowable while the deformation of the reinforce member 300 due to the axial external force may be sufficiently restrained. Particularly, the shape variations along the length L of the reinforce member 300 such as the curved surface and the wave shaped surface thereof may accelerate the deformation of the reinforce member 300 due to the radial external force, to thereby reduce resistivity to the radial external force. Therefore, the flexibility of the external sheet 200 may not be deteriorated in spite of the reinforce member 300. In contrast, when the shape variations along the width W of the reinforce member 300 may improve the resistivity to the deformation of the reinforce member 300 due to the axial external force, to thereby improve the strength of the external sheet 200 along the axis of the hollow shaft 100.

Accordingly, the external sheet 200 may be sufficiently prevented from being turned inside out by the axial external force and the deformation of the external sheet 200 by the radial external force may be freely allowable in spite of the reinforce member 300. Therefore, the reinforce member 300 may sufficiently improve the strength of the external sheet 200 in the axial direction of the hollow shaft 100 without any deterioration of the flexibility of the external sheet 200 in the radial direction of the hollow shaft 100. That is, the hollow shaft 100 may be prevented from being exposed to surroundings due to the turning inside out of the external sheet 200 caused by the axial external force.

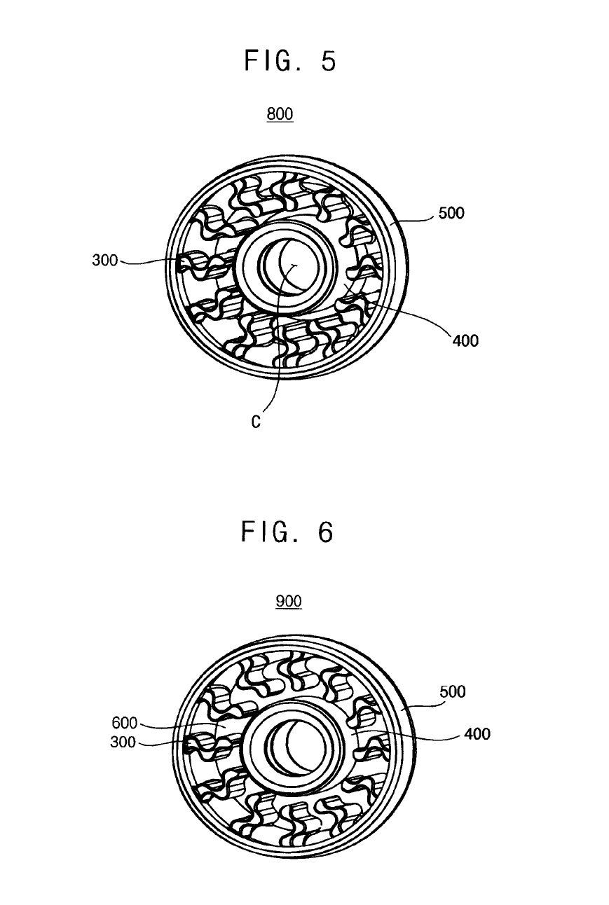

FIG. 5 is a perspective view illustrating an ear tip in accordance with another example embodiment of the present inventive concept. The ear tip 800 in FIG. 5 may have the same structure and configuration as the ear tip 700 shown in FIG. 1A, except for the materials of the hollow shaft and the external sheet. Thus, in FIG. 5, the same reference numeral denotes the same elements in FIG. 1A and any further detailed descriptions on the same elements will be omitted hereinafter.

Referring to FIG. 5, an ear tip 800 in accordance with another example embodiment of the present inventive concept may include a hard cylindrical hollow shaft 400 having a sound conduit C through which audio signals may be transferred, a soft external sheet 500 extending from an end portion of the hard hollow shaft 400 and enclosing the hard hollow shaft 400 in such a configuration that a gap space S may be provided between the hard hollow shaft 400 and the soft external sheet 500 and a reinforce member 300 protruded from the soft external sheet 500 toward the hard hollow shaft 400 in the gap space S to thereby reinforce a strength of the soft external sheet 500 and to sufficiently resist against an axial external force applied to the soft external sheet 500 along the hard hollow shaft 400. The material property terms of ‘hard’ and ‘soft’ are used relatively with respect to interrelations of the hollow shaft 400 and the external sheet 500, and are not defined on absolute bases.

The external sheet 500 may be inserted into the ear hole and make contact with the skin in the user's ear, thus the flexibility rather than hardness may be required to the external sheet 500 so as to improve adaptability of the ear tip 800 in the ear hole. In contrast, since the sound or the audio signals may be transferred to the ear through the sound conduit C, the sound wave may be required to be well reflected from an inner surface of the sound conduit C. For that reason, the hardness rather than the flexibility may be required to the hollow shaft 400 so as to improve the quality of the sound through the ear phone. Therefore, the external sheet 500 and the hollow shaft 400 may have contradictory material properties.

In the present example embodiment, the hollow shaft 400 may comprise relatively hard materials and the external sheet 500 may comprise relatively soft materials, and thus the functions of the hollow shaft 400 and the external sheet 500 may be maximized, respectively, to thereby improve the sound transfer characteristics and the comfort and stability of the ear tip 800 in the user's ear.

For example, the hard hollow shaft 400 may comprise silicon having the hardness of about 50° to about 60°, and the soft external sheet 500 may comprise silicon having the hardness of about 10° to about 15°. Thus, the hard hollow shaft 400 and the soft external sheet 500 may be individually formed by respective processes. In addition, any other materials may be used for the hollow shaft 400 and the external sheet 500 as long as the hardness of the material may be sufficiently varied in some ranges.

The reinforce member 300 may have the same material as the soft external sheet 500 and may be integrally formed together with the soft external sheet 500. Otherwise, the reinforce member 300 may comprise materials different from the soft external sheet 500 and may be formed on the inner surface of the soft external sheet 500 in an additional process. For example, the reinforce member 300 may comprise silicon having the hardness of about 50° to about 60°, and the reinforce member 300 may comprise silicon having the hardness of about 25° to about 40°.

The hard hollow shaft 400 may also include the guide unit 110 for guiding a connector unit (not illustrated) of the ear phone body to the hollow shaft 100, the stepped unit 120 to which the connector unit of the ear phone body may be coupled to thereby prevent the separation of the hollow shaft 100 and the connector unit and a conduit body 130 for transferring the audio signals into the user's ear, just like the hollow shaft 100 of the ear tip 700 shown in FIG. 1A. Thus, any detailed descriptions on the configurations of the hard hollow shaft 400 are omitted.

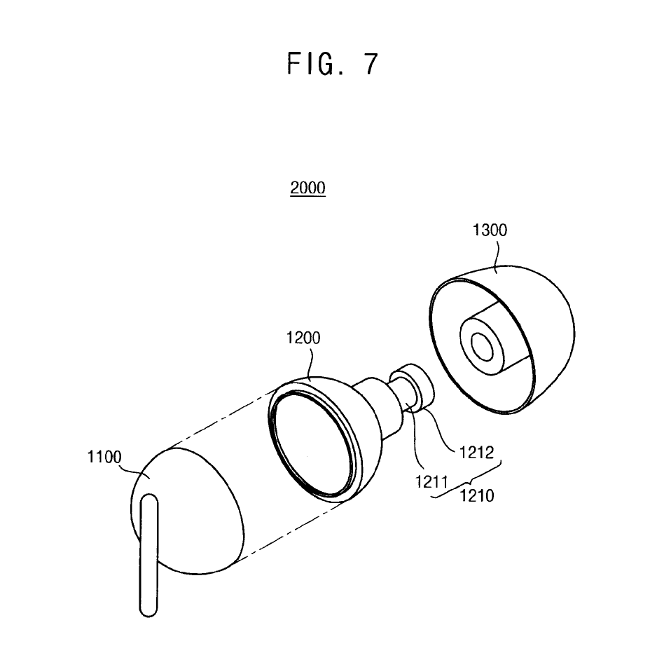

FIG. 6 is a perspective view illustrating an ear tip in accordance with still another example embodiment of the present inventive concept. The ear tip 900 in FIG. 6 may have the same structure and configuration as the ear tip 800 shown in FIG. 5, except for a noise absorber in the gap space S. Thus, in FIG. 6, the same reference numeral denotes the same elements in FIG. 5 and any further detailed descriptions on the same elements will be omitted hereinafter.

Referring to FIG. 6, an ear tip 900 in accordance with still another example embodiment of the present inventive concept may include a hard cylindrical hollow shaft 400 having a sound conduit C through which audio signals may be transferred, a soft external sheet 500 extending from an end portion of the hard hollow shaft 400 and enclosing the hard hollow shaft 400 in such a configuration that a gap space S may be provided between the hard hollow shaft 400 and the soft external sheet 500, a reinforce member 300 protruded from the soft external sheet 500 toward the hard hollow shaft 400 in the gap space S to thereby reinforce a strength of the soft external sheet 500 and to sufficiently resist against an axial external force applied to the soft external sheet 500 along the hard hollow shaft 400, and a noise absorber 600 arranged in the gap space S and absorbing the environmental noises to thereby preventing the noise being transferred into the user's ear. The material property terms of ‘hard’ and ‘soft’ are also used relatively with respect to interrelations of the hollow shaft 400 and the external sheet 500, and are not defined on absolute bases.

The hardness of the hollow shaft 400 may be helpful to transfer the high frequency audio signals. However, the environmental noises may also be much more transferred into the ear due to the hardness of the hollow shaft 400. Particularly, since the softness of the external sheet 500 may improve the sealing between the external sheet 500 and the skin of the ear and thus the gap between the external sheet 500 and the ear skin may be almost reduced, the noise may be transferred into the ear through the hollow shaft 400 rather than the gap between the external sheet 500 and the ear skin. Since the sound wave is generally transferred through a hard medium much more than through a soft medium due to the wave characteristics, it is expected that the noise may be transferred into the ear through the hard hollow shaft 400 much more than the soft external sheet 500.

For those reasons, the noise absorber 600 may be provided at a bottom of the pot-shaped external sheet 500 in such a configuration that the hollow shaft 400 may be enclosed by the noise absorber 600 around the first end portion of the hollow shaft 400. The noise from the outsides of the ear may be transferred into the gap space S between the hollow shaft 400 and the external sheet 500 and may be absorbed into the noise absorber 600. Thus, the noise may be blocked off at the bottom of the hollow shaft 400 and may not be transferred into the ear. In the present example embodiment, the noise absorber 600 may comprise a soft material having a low hardness for increasing absorption efficiency of the noise. For example, the noise absorber 600 includes an elastic gel-state silicon based material having the hardness of about 5° to about 20°. Particularly, silicon gel may have an excellent resilience property and the deformation of the silicon gel caused by an external force may be easily restored to an original shape. That is, the silicon gel may have excellent flexibility and thus may be easily deformed by the external force as well as the deformation may be easily restored to the original shape in removing the external force to the silicon gel.

Therefore, when the noise absorber may include the silicon gel, both of the soft external sheet 500 and the noise absorber 600 may be easily deformed by the radial external force and may also be easily restored to the original shape when removing the radial external force due to the high flexibility of the silicon gel. Accordingly, the ear tip 900 may be compressed and reduced into a size smaller than the ear hole in accordance with the radial external force applied by the user and then may be inserted into the user's ear hole. After the ear tip 900 may be inserted into the user's ear hole, the radial external force may be removed and the soft external sheet 500 and the noise absorber 600 may be rapidly restored in harmonious with the shape of the user's ear hole due to the resilience of the external sheet 500 and the noise absorber 600. The restoration in accordance with the user's ear hole may sufficiently improve the contact stability and sealing between the ear tip 900 and the ear skin.

Accordingly, the noise absorber 600 may sufficiently prevent the noise transfer into the ear through both of the noise absorption and high sealing between the ear tip and the ear skin due to the elastic resilience of the noise absorber 600. Thus, the high quality sound may be transferred into the user's ear without any noises to thereby improve the performance of the ear phone including the ear tip 900.

While the present example embodiment discloses the noise absorber in the ear tip in which the hollow shaft and the external sheet may have different materials as illustrated in FIG. 5, the noise absorber may also be provided with the ear tip in which the hollow shaft and the external sheet may have the same materials as illustrated in FIG. 1A, as would be known to one of the ordinary skill in the art.

FIG. 7 is an explosive perspective view illustrating an ear phone including an ear tip in accordance with an example embodiment of the present inventive concept.

Referring to FIG. 7, an ear phone 2000 in accordance with an example embodiment of the present inventive concept may include a housing 1100 including an audio signal generator (not illustrated), a cover 1200 detachably coupled to the housing 1100 such that the housing 1100 is covered with the cover 1200 and an inner space of the housing 1100 is isolated from surroundings and an ear tip detachably attached to the cover 1200 and guiding the audio signals into the user's ear.

For example, a transducer for transforming electronic/electrical signals into the audio signals may be arranged in the housing 1100 and thus the audio signals may be generated by using vibrations of metal plates. The electronic/electrical signals may be transferred to the transducer by various sound systems. Various drivers such as a permanent magnet, an electromagnet and a piezoelectricity device may be further arranged in the housing 1100 in accordance with the sound quality.

The cover 1200 may be detachably coupled to the housing 1100 and thus the inner space of the housing 1100 may be sealed from surroundings. The audio signal generator including transducer and the driver may be secured in the housing 1100 by the cover 1200. A sound guide 1210 may be installed to the cover 1200 and the sound or the audio signals in the housing 1100 may be discharged out of the closed inner space of the housing 1100. For example, the sound guide 1210 may be shaped into a tube and may include a recess 1211 and a protrusion 1212 that may be coupled to the ear tip 1300 at an end portion thereof.

The ear tip 1300 may be detachably coupled to the cover 1200 in the medium of the sound guide 1210 and may be inserted into the user's ear hole, and thus the contact stability of the ear phone to the skin of the user's ear may be improved by the ear tip 1300. In addition, the ear tip 1300 may prevent the noise transfer into the ear through both of the high quality of sealing and noise absorption. The ear tip 1300 may have the same structures and configurations as described in detail with reference to FIGS. 1A to 6, and thus any further detailed descriptions on the ear tip 1300 is omitted.

Particularly, the protrusion 212 of the sound guide 1210 may be coupled to the stepped unit 120 via the guide unit 110 of the hollow shaft 100, and thus the sound guide 1210 of the cover 1200 may be stably coupled to the hollow shaft 100. Since the guide unit 110 may be shaped into the trapezoidal cylinder, the sound guide 1210 may be smoothly inserted into the guide unit 110 just by a user's push. The stepped unit 120 may have a receiving space of which the size may be greater than the guide unit 110, and thus the protrusion 1212 may be sufficiently received in the receiving space of stepped unit 120. Therefore, once the protrusion 1212 may be positioned in the receiving space of the stepped unit 120, the stepped unit 120 and the protrusion 1212 may make surface contact with each other along the axial direction of the hollow shaft 100, to thereby improve coupling reliability between the sound guide 1210 and the hollow shaft 100 of the ear tip 1300.

When the ear phone 2000 including the ear tip 1300 may be removed from the user's ear, the axial external force may be applied to the external sheet 200 of the ear tip 1300. However, since the strength of the external sheet 200 may be sufficiently reinforced by the reinforce member 300 along the axial direction of the hollow shaft 100, the external sheet 200 of the ear tip 1300 may be sufficiently prevented from being turned inside out in removing the ear phone from the user's ear. In addition, the noise absorber 600 in the ear tip 1300 may improve the contact stability of the ear phone 2000 with respect to the skin of the user's ear and may prevent the noise transfer into the ear through both of the high quality of sealing and noise absorption.

According to example embodiments of the present inventive concept, the reinforce member may be provided in the gap space S between the hollow shaft including the sound conduit and the external sheet enclosing the hollow shaft, and thus the strength of the external sheet may be reinforced by the reinforce member along the axial direction of the hollow shaft. Accordingly, the external sheet may be prevented from being turned inside out by the axial external direction, to thereby prevent the breakage of the ear tip and increase the durability of the ear tip. In addition, the noise absorber may be further provided around the bottom of the hollow shaft in the gap space, to thereby efficiently prevent the noise transfer into the ear and increase sound quality. Particularly, when the hollow shaft of the ear tip may comprise a relatively hard material and the external sheet of the ear tip may comprise a relatively soft material, both of the contact stability and the sound quality of the ear phone may be improved due to the material properties of the ear tip.

The foregoing is illustrative of example embodiments and is not to be construed as limiting thereof. Although a few example embodiments have been described, those skilled in the art will readily appreciate that many modifications are possible in the example embodiments without materially departing from the novel teachings and advantages of the present invention. Accordingly, all such modifications are intended to be included within the scope of the present invention as defined in the claims. In the claims, means-plus-function clauses are intended to cover the structures described herein as performing the recited function and not only structural equivalents but also equivalent structures. Therefore, it is to be understood that the foregoing is illustrative of various example embodiments and is not to be construed as limited to the specific example embodiments disclosed, and that modifications to the disclosed example embodiments, as well as other example embodiments, are intended to be included within the scope of the appended claims.

What is claimed is:

1. An ear tip for an ear phone, comprising:
a cylindrical hollow shaft having a sound conduit through which audio signals are transferred into a user's ear;
an external sheet extending from an end portion of the hollow shaft and enclosing the hollow shaft such that a gap space is provided between the hollow shaft and the external sheet; and
a reinforce member protruded from the external sheet toward the hollow shaft in the gap space and reinforcing a strength of the external sheet to thereby resist against an axial external force applied along the hollow shaft.

2. The ear tip of claim 1, wherein the hollow shaft, the external sheet and the reinforce member include same materials.

3. The ear tip of claim 2, wherein the hollow shaft, the external sheet and the reinforce member comprise silicon having a hardness of 20° to 45°.

4. The ear tip of claim 1, wherein the reinforce member includes one of the curved plate, a flat plate a cone and a waved plate that is protruded from an inner surface of the external sheet.

5. The ear tip of claim 1, wherein the hollow shaft comprises a material different from the external sheet and the hollow shaft and the external sheet are individually connected to each other.

6. The ear tip of claim 5, wherein the hollow shaft comprises silicon having hardness of 50° to 60° and the external sheet comprises silicon having hardness of 10° to 15°.

7. The ear tip of claim 6, wherein the reinforce member comprises silicon having hardness of 20° to 45°.

8. The ear tip of claim 1, further comprising a noise absorber arranged around the end portion of the hollow shaft in the gap space, the noise absorber absorbing environmental noises and preventing noise transfer into the user's ear.

9. The ear tip of claim 8, wherein the noise absorber includes an elastic gel state material having hardness of 5° to 20°.

10. An ear phone comprising:
a housing including an audio signal generator;
a cover detachably coupled to the housing such that the housing is covered with the cover and an inner space of the housing is closed from surroundings, the cover including a sound guide through which the audio signal is discharged out of the closed inner space; and
an ear tip detachably attached to the cover and guiding the audio signals into the user's ear;
wherein the ear tip includes a cylindrical hollow shaft having a sound conduit through which the audio signals are transferred into a user's ear, an external sheet extending from an end portion of the hollow shaft and enclosing the hollow shaft such that a gap space is provided between the hollow shaft and the external sheet, and a reinforce member protruded from the external sheet toward the hollow shaft in the gap space and reinforcing a strength of the external sheet to thereby resist against an axial external force applied along the hollow shaft.

11. The ear phone of claim 10, wherein the sound guide is shaped into a tube having at least a recess and at least a protrusion at an end portion thereof and the hollow shaft of the ear tip includes a stepped unit having a receiving space in which the protrusion is received in such a configuration that the protrusion and the stepped unit makes surface contact with each other in an axial direction of the hollow shaft.
